(19) United States
(12) Patent Application Publication
(10) Pub. No.: US 2006/0235314 A1
(43) Pub. Date: Oct. 19, 2006

(54) Medical and surgical devices with an integrated sensor

(76) Inventors: Michele Migliuolo, Pittsburgh, PA (US); William D. Suh, Cary, NC (US); Albert P. Pisano, Danville, CA (US); Doran Liepmann, Lafayette, CA (US); Kyle Lebouitz, Pittsburg, PA (US); Jennifer Rogers, Pittsburg, PA (US); Mordechay Schlesinger, Pittsburg, PA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/543,739

(22) PCT Filed: Jan. 30, 2004
(86) PCT No.: PCT/US04/02547

§ 371 Date: Apr. 21, 2006

Related U.S. Application Data
(60) Provisional application No. 60/443,877, filed on Jan. 31, 2003.

(51) Int. Cl. A61B 5/02; A61B 5/00

(52) U.S. Cl. 600/505; 600/549; 600/561

(57) ABSTRACT

Medical or surgical instrument, such as a catheter, micro-catheter, guide-wire, cannula, blade, and forceps, comprising one or more sensors to allow measurement of parameters around or within the instrument, and methods for making and using the same, are disclosed. Also disclosed are methods and apparatuses for measuring fluid characteristics, such as blood characteristics, including, for example, velocity, flow direction, and pressure in a vascular system.

### PRIORITY DATA

U.S. Provisional Patent Application No. 60/443,877 (Jan. 31, 2003).

### DESCRIPTION OF THE INVENTION

1. Field of the Invention

The present invention relates to a medical or surgical instrument, such as a catheter, micro-catheter, guide-wire, cannula, blade, and forceps, comprising one or more sensors to allow measurement of parameters around or within the instrument, and methods for making and using the same. The present invention also relates to methods and apparatuses for measuring fluid characteristics, such as blood characteristics, including, for example, velocity, flow direction, and pressure in a vascular system. A measuring device incorporated at or near the distal end of a catheter system can be used in, for example, angioplasty, coronary vessel diagnostics, coronary stent delivery, cancerous tumor treatment, insulin islet infusions, the measurement of angiographic pressure/flow, or any other medical catheter-based methods. According to certain embodiments, the present invention also relates to measuring blood characteristics at a single location within a vascular system, at multiple locations to provide a profile to yield, for instance, a blood velocity profile along a length in the blood vessel, or at any combination thereof.

2. Background

The measurement of physiological parameters such as temperature, flow rate, flow direction, density, force, temperature, pH, biochemical composition (e.g., antigens, pathogens, antibodies, viscosity, complete blood count (CBC), ions such as sodium, potassium, and calcium, and gasses such as O₂ and CO₂), location, size, distance, and pressure, is of interest for medical diagnosis in procedures such as angioplasty, coronary vessel diagnostics, coronary stent delivery, cancerous tumor treatment, insulin islet infusions, the measurement of angiographic pressure/flow, or any other medical catheter-based methods. The measurement of instrument parameters, such as temperature, contact, force, strain, location, velocity, and acceleration is likewise of interest for medical and surgical applications. Measurements of physical parameters such as these physiological and instrument parameters can be made using (microelectromechanical systems) MEMS-sensors. However, available MEMS-sensors are discrete units that may be difficult to integrate into medical tools.

With the advent of MEMS technology, especially using silicon, surgical tools constructed entirely of semiconductor materials, such as silicon, having the ability to sense, for example, temperature or strain, are known. Examples of such devices are described in Carr et al., U.S. Pat. No. 5,980,518, entitled “Microcautery Surgical Tool,” and Mehregany et al., U.S. Pat. No. 5,579,583, entitled “Microfabricated Blade,” both of which are incorporated herein by reference. These types of semiconductor material devices may allow a possible direct integration of circuitry in the MEMS device. However, semiconductor materials such as silicon tend to be brittle and not always well suited for use as the primary structural component of surgical, industrial, and many consumer devices. They are also not ideally suited for use on curved surfaces, since semiconductor processing methods usually involve and yield devices on flat surfaces that cannot be conformed to the profile of a curved surface.

In a broader perspective, the aforementioned sensors and their associated fabrication methods are not always suitable for integrating sensors onto surgical instruments, especially given a two-step integration process: (1) fabricating of the sensor itself on the material other than the surgical instrument itself, such as on a silicon substrate, and (2) integrating the sensors into or onto the surgical instrument by hand or machine with epoxy, tape, or some other form of attachment or adhesion.

Forming sensors in this manner on medical or surgical devices such as catheters can have certain deficiencies, including that as sensors, heaters, and any sensor related features are not easily mounted on catheters. Their mounting typically involves manual handling that increases integration time, increase associated cost, and decreases reliability. Also, the addition of such components may increase the overall diameter of the catheter and may change the flexibility of the catheter. Factors such as these can constrain where the device can be placed, and make the device less useful and less reliable for application in various small diameter openings and friction surfaces during a medical procedure. For example, maintaining original size and flexibility of a catheter would avoid re-enigineering of catheter itself, and would avoid any unnecessary and undesirable changes to their manipulation in a given procedure.

Accordingly, a practical need exists in the art for a better method of integrating sensors on surgical instruments, including on instruments such as catheters that have curved outer surfaces. An object of the present invention is to provide a method of forming sensors on surgical instruments which overcomes at least one of the mentioned shortcomings. Another object is a surgical instrument, such as a catheter, that includes sensors for measuring properties such as fluid flow, such as blood velocity, and methods of making and using the same.

### SUMMARY

According to certain embodiments, the present invention relates to sensors directly integrated on a surgical instrument, and methods of making the same. The sensors may be configured to measure, for example, physiological parameters such as temperature, flow rate, flow direction, density, temperature, location, size, distance, and pressure, and instrument parameters, such as temperature, contact, strain, location, velocity, and acceleration is likewise of interest. For example, according to certain embodiments there is a device for measuring blood flow in a blood vessel, that includes a surgical instrument such as a catheter that has a curved outer surface and at least one conformal blood flow sensor on the curved outer surface. According to this embodiment, the at least one blood flow sensor is configured to measure the blood flow, and may be configured to measure other parameters as well.

According to certain embodiments, a surgical instrument, such a catheter, serves as a substrate on which sensors and optionally other components and layers may be fabricated. According to certain embodiments, the substrate does not need to be a semiconductor material, but may be, for example, an insulator such as a polymer or a conductor such as a metal. One or more conformal sensors may be fabricated directly on the substrate using thin-film depositions with a conformal shadow masking to define the features. Additionally, according to certain embodiments, conformal sensors may be precisely positioned on a surgical instrument and may better follow the contours of the substrate without manual handling during the sensor integration.

According to certain embodiments, the fabrication method eliminates the necessity of any post processing glue layers and handling associated with current methods of sensor integration, though such steps may be included or excluded depending upon the particular embodiment. Fabrication methods according to certain embodiments of the present invention may be used to form sensors on surgical instruments in a final or near-final product form without substantially interfering with or changing the instrument's original functions, shapes, or both.

Conductive traces, such as for power or information transfer in the operation of a sensor, can also be formed on the surgical instruments by using one or more techniques according to certain embodiments of the present invention. In certain cases, conductive traces already embedded in a surgical instrument can be utilized. For example, conductive traces or wires may be helically wound or longitudinally laid on or within the exterior wall of a catheter. Conductive wires can also be passed through an interior channel of the instrument, such as a catheter lumen.

Electrical contacts among features such as wires, sensors and control circuits can be made, for example, through access opening on a surgical instrument by making selective exposures of the wires that are covered by the instrument itself (when embedded within the substrate) or by other protective layers. The exposure can be achieved by, for example, the application of localized, ablation and/or etching on certain locations of the instrument where the wires are to be exposed. After access openings are made, sensors and other necessary and optional layers may be directly formed over them to make contact with wires. By varying the number and location of access openings, the integration of various sensors at one or more locations on a surgical instrument is possible.

According to certain embodiments of the present invention, there is an industrially practical manufacturing method that minimizes or eliminates the manual assembly of mechanical or electrical components. The sensor material's stoichiometry, geometry and thermal and electrical characteristics can also be controlled in order to make them suitable for various surgical instruments in different operating conditions.

Further features and advantages of the present invention, together with additional objects and advantages thereof, will be apparent upon consideration of the following detailed description, taken in conjunction with the following drawings.

### BRIEF DESCRIPTION OF THE DRAWINGS

Figs. 1A-1C are perspective views of several embodiments of a single lumen catheter according to the present invention;

FIG. 2 is a perspective view of an embodiment of a multi-lumen catheter according to the present invention;

FIG. 3A is a perspective view of another embodiment of a multi-lumen catheter according to the present invention;

FIG. 3B is a cross-sectional view of the catheter of FIG. 3A taken along line 3B-3B;

FIG. 4A is a perspective view of another embodiment of a single lumen catheter according to the present invention;

FIG. 4B is a cross-sectional view of the catheter of FIG. 4A taken along line 4B-4B;

FIG. 5 is a perspective view of another embodiment of a single lumen catheter according to the present invention having a layer on its outer surface;

FIG. 6A is a perspective view of another embodiment of a single lumen catheter according to the present invention having a layer on its outer surface;

FIG. 6B is a cross-sectional view of the catheter of FIG. 6A taken along line 6B-6B;

FIG. 7A is a side perspective view of an embodiment of a single lumen catheter according to the present invention having sensors connected in series by electrically conductive layers;

FIG. 7B is a top perspective view of the catheter of FIG. 7A;

FIG. 8A is a side perspective view of an embodiment of a single lumen catheter according to the present invention having a sensor layer;

FIG. 8B is a top perspective view of the catheter of FIG. 8A;

FIG. 8C is a perspective view of another embodiment of a single lumen catheter according to the present invention having a sensor layer;

FIG. 8D is a perspective view of an embodiment of a single lumen catheter according to the present invention having multiple sensors;

FIG. 9A is a side perspective view of an embodiment of a single lumen catheter according to the present invention having a sensor layer and a protective coating;

FIG. 9B is a top perspective view of the catheter of FIG. 9A;

FIG. 10 is a flow chart outlining processing steps according to certain embodiments of the present invention;

Fig. 11A is a detail view of an embodiment of a mask used for the deposition of the insulation layer shown in FIG. 5;

FIG. 11B is a detail view of an embodiment of a mask used for the deposition of the contact layer shown in FIG. 6;

FIG. 11C is a detail view of an embodiment of a mask used for the deposition of the sensor layer shown in FIGS. 8A and 8B;

FIG. 11D is a detail view of an embodiment of a mask used for the deposition of the protection layer shown in FIGS. 9A and 9B;

FIGS. 12A-12C are schematic views of embodiments of substrate fixtures according to the present invention;

FIG. 13 is a schematic view illustrating an embodiment of a plating method according to the present invention;

FIGS. 14A and 14B are perspective views illustrating the attachment of a sensor according to the present invention to a surgical instrument;

FIG. 15 is a chart illustrating pulsed width modulation (PWM) signals that can be used to heat a flow sensor;

FIG. 16 is a perspective view of an embodiment of a single lumen catheter according to the present invention having two sensors;

FIG. 17 is a perspective view of an embodiment of a single lumen catheter according to the present invention having three sensors;

FIG. 18 is a schematic view illustrating a method of detecting a partial blockage in a blood vessel using a catheter having multiple sensors;

FIG. 19A is a side perspective view of an embodiment of a single lumen catheter according to the present invention having a piezoelectric ultrasonic sensor layer and a protective coating;

FIG. 19B is a top perspective view of the catheter of FIG. 19A;

FIG. 20 is a schematic view illustrating a method of simultaneously detecting blood pressure, blood flow, and blood vessel blockage using a catheter having multiple sensors;

FIG. 21 is a schematic of a sensor control and test system;

FIGS. 22A-B are graphs of measured sensor output as functions of fluid flow rates; and

FIG. 23 is a graph of a measure sensor output as a function of local tissue temperature.

### DESCRIPTION OF THE CERTAIN EMBODIMENTS OF THE INVENTION

As used herein, the term “flat,” as used with respect to a surface, indicates the surface is not curved, and will be considered “flat” notwithstanding any sharpened edges or any slight curvature. An example of a flat surface is the surface perpendicular to the cutting edge of a standard razor blade, such as a Gillette® Super Stainless Super Inoxydable blades. In contrast, the term “curved,” as used herein with respect to a surface, indicates that the surface is non-flat. The outer surfaces RENEGADE® HI-FLO (Boston Scientific, Inc) and FOGARTY™ VENOUS THROMVECTOMY (Edwards Lifesciences Corporation) catheters are examples of non-flat surfaces. As another example, a curved surface will have a radius of curvature of less than 20 cm, such as less than 10 cm, such as less than 5 cm, such as less than 1 cm, such as less than 5 mm, such as less than 1 mm, such as less than 0.5 mm, such as less than 0.25 mm, such as less than 0.1 mm, over the portion of the surface in question. The change in profile may be, for example, generally smooth (such as a circular profile), though not necessarily at a uniform rate (such as an elliptical profile). The portion of the surface in question may be, for example, the portion covered by one or more sensor components or the portion covered or partially covered by a conformal shadow mask during a thin film deposition step. Additionally, a curved surface may contain one or more substantially flat subsections, such as a where a portion of the outer surface of a cylinder has been truncated beyond a plane parallel to the cylinder's central axis. The curved surface may also contain one or more recesses or raised features, and still be considered curved.

As used herein, “depositing” or “forming” a layer on a surface or features indicates that the layer is applied to or formed on the surface or feature either directly, i.e., with no interposing layers or features, or with other layers and/or features interposed between the deposited layer and the surface or feature. If the layer is necessarily deposited directly on a surface or feature, in the absence of any interposing layer or feature, this will be referred to as a layer “directly deposited” on the surface or feature, or with grammatical variants of this term. Unless otherwise specified, the layers referred to herein may be directly deposited or deposited with one or more interposing layers or features. It should also be understood that an interposed layer or feature may, in some cases, be only interposed between select portions of a given layer and the underlying surface. According to certain embodiments, a given features may be directly deposited on an indicated substrate, feature, or layer.

As used herein, a “layer” indicates a deposited coating or feature over some part, but not necessarily all, of a surface. For example, a conductive trace deposited on select locations on a surface would be considered a deposited layer. It would be considered as deposited on the surface notwithstanding any interposed layers or features, such as an insulation layer, unless referred to as a “directly deposited” layer, as explained above.

As used herein, a “conformal” sensor is a sensor that is integrated into the underlying device (such as a catheter, for a sensor integrated on a catheter), where the underlying device serves as a substrate for the formation of some or all the features of the sensor, and the sensor conforms with the surface profile, be it flat or curved. A conformal sensor in this context is distinct from a rigid sensor, such as a rigid sensor formed on or from a semiconductor wafer, that is merely affixed to the underlying device after the sensor is fully formed separately from the underlying device, and which cannot be conformed to the shape of the surface, such is described in Lebouitz et al., U.S. Pat. No. 6,494,882, column 7, line 50 to column 8, line 38. The addition of one or more conductive traces for providing power to or information to or from a separately formed flat, rigid sensor applied to a flat surface does not convert the rigid sensor into a conformal sensor, as it is understood in the present context. However, a sensor resulting from the formation of integrated features on a substrate that are connected to an attached sensor element (or sub-element) formed separately on a flexible substrate that can be conformed to the profile of the surface is considered one type of a conformal sensor.

Fabrication of sensors and other elements, such as conformal sensors, on flat and non-flat surfaces can be achieved through the use of techniques such as flexible shadow masking, laser ablation, and 3-D thin-film deposition. Specifics of each technique will be explained after first describing a general fabrication process.

Micro sensors can be fabricated for sensing applications such as measurements of strain, pressure, temperature, density, distance, the presence of objects such as nerves, and movement. For example, a strain sensor or gauge can be constructed using a resistor made of a material such as polysilicon. The resistance of a material such as polysilicon changes as it is stretched or compressed, and by measuring the change in resistance, one can calculate the strain. As another example, a pressure sensor can be constructed by placing a strain sensor on top of a diaphragm made of a deformable material such as silicon nitride or polysilicon. When the diaphragm moves (i.e., expands or contracts) due to surrounding pressure changes, the strain gauge can be used to measure the local pressure. Examples of such pressure sensors are described in S. Sugiyama et al., “Micro-diaphragm Pressure Sensor,” IEEE Int. Electron Devices Meeting, pp. 184-7 (1986), and H. Tanigawa et al., “MOS Integrated Silicon Pressure Sensor,” IEEE Trans. Electron Devices, Vol. ED-32, No. 7, pp. 1191-5 (July 1985), the disclosures of which are incorporated hereby by reference.

Similar to a strain sensor, a temperature sensor can be constructed using a resistor made of a material such as polysilicon having a temperature dependent electronic property, such as its resistivity. Using a sensor of this type, temperature can be measured as a function of the change in, for example, the resistance of the material. Further, as described in A. S. Sedra and K. C. Smith, “Microelectronic Circuits,” 4^{th} Ed., Oxford University Press, New York, p. 135 (1998), the disclosure of which is incorporated herein by reference, diodes can be used to measure temperature dependence and thus may also be used in temperature sensors.

Piezoelectric ultrasonic sensors can be used to measure, for example, density. Such sensors vibrate at a high frequency and can emit in the direction of the object of interest an ultrasonic signal. Density of the impinged object can then be measured based on the ultrasonic signal that is reflected back by that object. Examples of such sensors are described in U.S. Pat. Nos. 5,129,262, and 5,189,914, and S. W. Wenzel and R. M. White, “A Multisensor Employing an Ultrasonic Lamb-wave Oscillator,” IEEE Trans. Electron Devices, Vol. 35, No. 6, pp. 735-743 (June 1988), the disclosures of which are incorporated herein by reference. Piezoelectric materials include polymeric piezoelectric material, piezoelectric ceramic materials, and composite. Specific examples of piezoelectric materials include polyvinylidene fluoride (PVDF), ZnO, PZT, PZN, and quartz.

The presence of nerve tissue can be detected using an electrical contact, such as a gold electrode, which picks up and conducts electrical signals in proximity therewith.

Blood flow velocity and direction can be measured by thin film anemometry techniques. Exemplary blood velocity sensors are disclosed in, for example, U.S. Pat. Nos. 3,359,974, 3,438,253, 4,841,981, 5,174,299, 5,271,408, 5,271,410, 5,373,850, 5,617,870, 5,799,350, 5,831,159, each of which is incorporated herein by reference. As disclosed in the above examples, sensor integration involves methods, such as inserting bulk sensors into catheter and wrapping wires or sheets over catheters or guide wires, that are not necessarily required according to certain embodiments of the present invention.

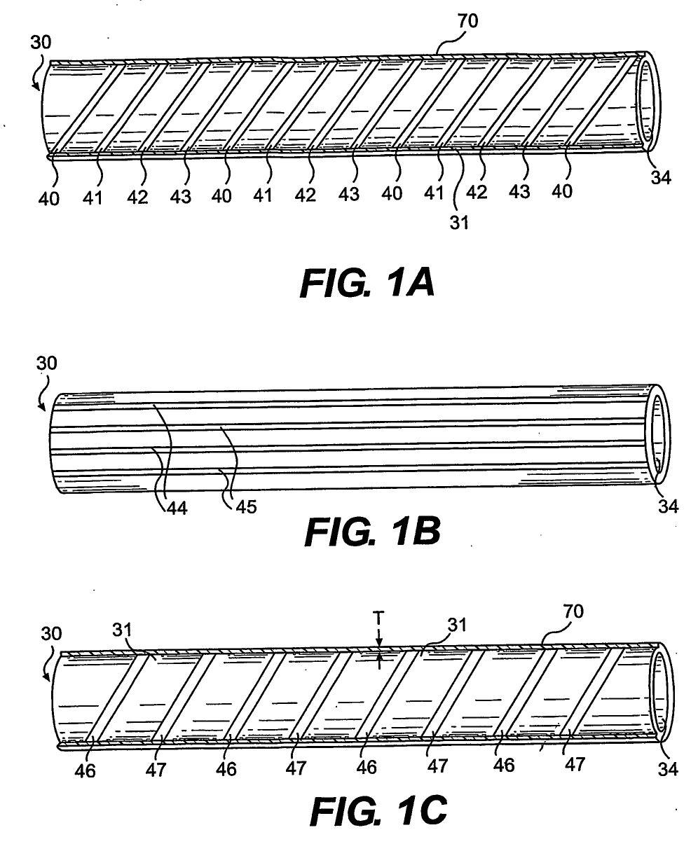

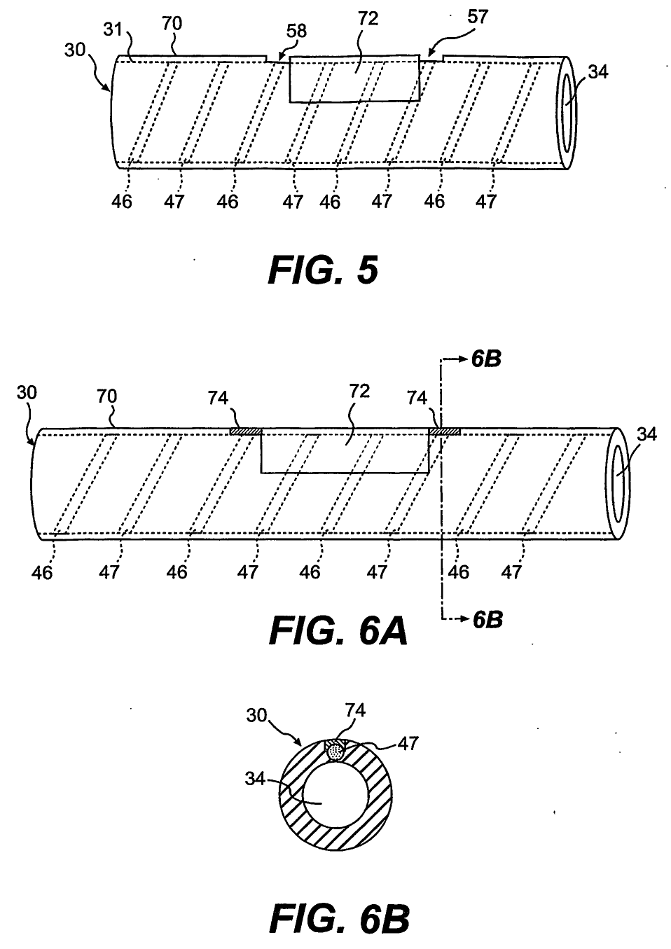

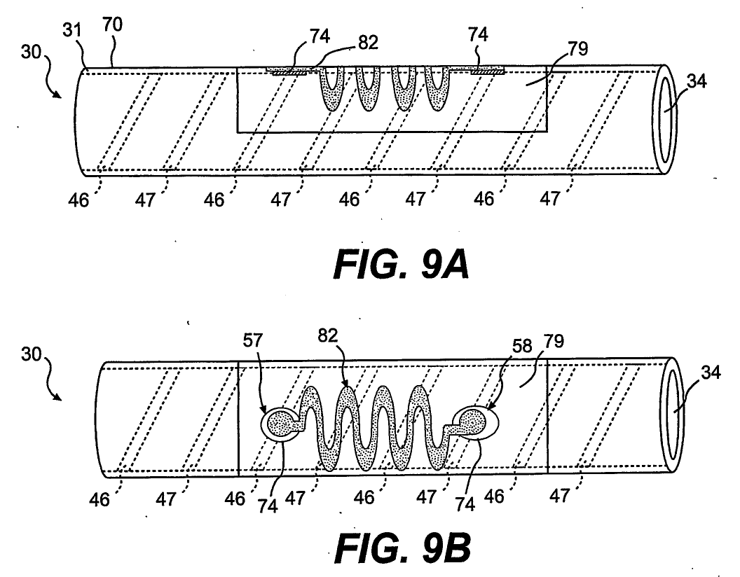

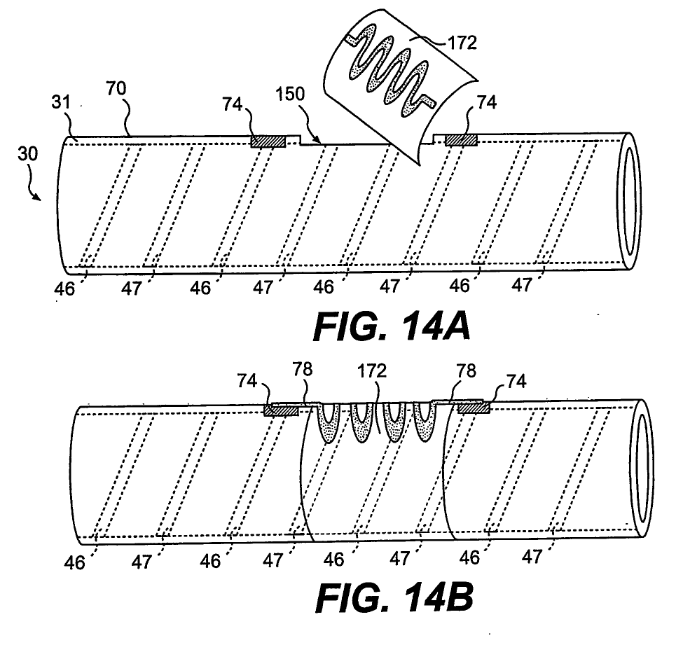

According to certain embodiments, the formation of a sensor may involve a combination of one or more of several basic steps. As illustrated in FIGS. 1-14 for the case of a catheter 30, these are making or otherwise incorporating conductive traces or wires 40-49 (see, e.g., FIGS. 1A-C, 2), making access openings or pockets 55, 56, 150 (see, e.g., FIGS. 3, 14A), depositing layers or features such as adhesion or insulation layers 72 (see, e.g., FIG. 5), depositing contact layers 74 (see, e.g., FIG. 6), depositing one or more sensor layers 78 or 78-76-78 (see, e.g., 7A), and depositing protection layers 79 (see, e.g., FIG. 9).

Orders and number (i.e., repetitions) of these steps are not limited to the presented orders and numbers, and not every step may be necessary for a given application. For purpose of illustration, the associated figures depict methods and devices of the present invention in connection with a blood velocity sensor on a catheter.

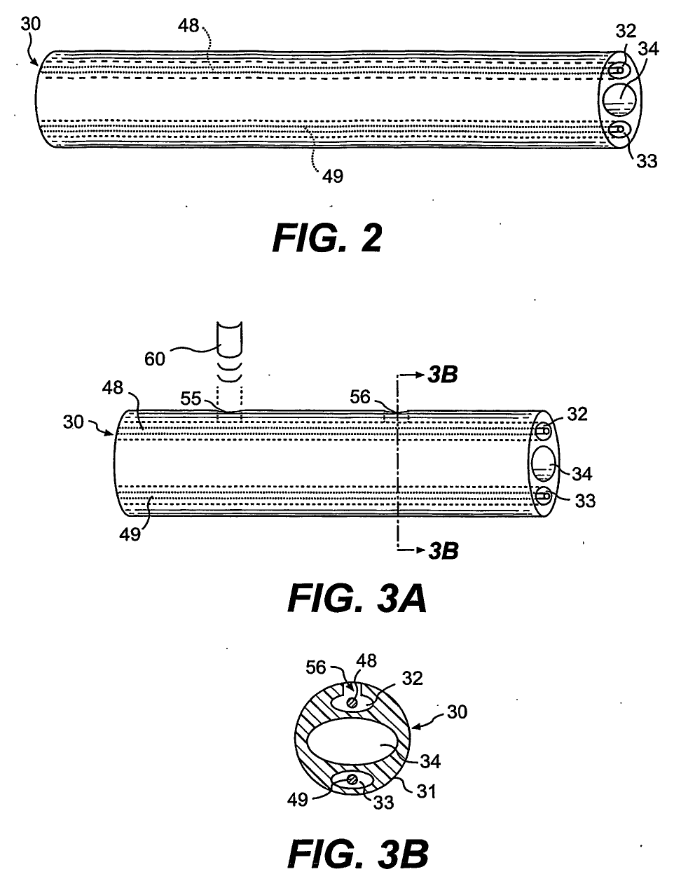

As illustrated in Figs. 1A-C a first step is to layout a wiring 40-47 on a surgical instrument, such as the exemplary catheter 30. Sensors that are formed directly on a surgical instrument may require wires or conductive traces for their power and/or information transfer. In certain embodiments, a surgical instrument, such as a catheter, in their final manufactured form may already have the embedded conductive traces, such as within their exterior wall. Examples of such catheters with embedded wires include RENEGADE® HI-FLO catheter (Boston Scientific, Inc) and FOGARTY™ VENOUS THROMVECTOMY catheter (Edwards Lifesciences Corporation). In such cases, utilization of the embedded conductive traces (wires) can minimize or eliminate this wiring step. FIGS. 1A-1C show partially transparent views (to show wires 40-47 within an outer wall or beneath and outer coating 70) of several exemplary wiring configurations that can be embedded within the exterior wall 31 of a catheter 30. Such wiring may be embedded through, for example, heat bonding of wires into a pre-formed catheter tube, or by extruding the catheter shaft with the wires in place. Wires 40-47 may also be fixed on the exterior wall of the catheter using heat-shrinkable tubing, such as optional outer layer 70, which has a thickness T. The optional outer layer 70 may be an added layer or may be coextensive with outer surface 31 The thickness of such wires will depend on the size of the device and its application, and may include, for example, commercially available thicknesses, such as from 0.0005″ to 0.002″. Wires 40-47 can also be thin conductive films. Wire spacing and the angles of helix winding can be adjusted to accommodate a different number of wires and their spacing relative to one another. Wires 44, 45 may also be embedded longitudinally, for example as shown in FIG. 1B. Wires 48, 49 may be pulled through lumens 32, 33 as depicted in FIG. 2. Thus, according to certain embodiments, at least one lumen 32, 33 in a multi-lumen catheter 30 is used to house at least one wire 48, 49. Additionally, conductive traces can be deposited directly on a substrate by the same or similar method explained in the steps of making the contact and sensor layers, presented below.

According to certain embodiments, a small area of wiring may be exposed, such as through an exterior catheter wall, to connect micro sensors, such as sensors for the measurement of at least one of blood temperature, velocity, flow direction, and pressure. Thus, as generally depicted in FIGS. 3A, B, a second step is to make access openings or pockets 55, 56 which provide access to the wiring 48, 49 or other layers and which may house sensors and other layers. As the terms are used herein, an access opening refers to a generally small hole for exposing layers or features underneath such as wires while a pocket refers to a generally larger recess that can contain sensors and/or other necessary layers. Depending on the types of application, one opening may be used as both a pocket and an access opening, or a pocket may contain one or more access openings.

Based on the material type of the substrate or any layers that require of access openings or pockets, suitable processes for chemical/physical etching or ablation of the substrate (i.e., exterior wall of catheter) may be chosen in consideration of the substrate's allowable temperature, chemical reaction, or any other mechanical restrictions. For example, a UV laser may be used to make an access opening on a polymer substrate, such as a polymer based catheter, without damaging the metal wire underneath because most of the UV energy is absorbed by the polymer. Also, the geometry of the access opening or pocket will be varied based on substrate (i.e., catheter) type, size, and the other restrictions. For example, a proportionally smaller or narrower access hole can be used for a catheter with small diameter and/or more flexibility in order not to change original stiffness by introducing the access opening(s).

According to certain embodiments, as illustrated in FIGS. 14A, B (discussed further below), for example, a pocket 150 is formed at the outer surface of a surgical tool (e.g. catheter 30) to improve the integration of a sensor assembly without increasing the original dimension of the surgical instrument. For instance, the resulting dimensions of a catheter's diameter do not need to be increased to accommodate the sensor assembly on its surface. A pocket 150 can be formed by, for example, chemical etching or laser ablation, of the catheter 30 and any coatings 70 or layers thereon. For example, laser ablation or focused ion beam (FIB) milling can be utilized for making an access opening or pocket on a low melting point substrate, such as certain polymer based catheters, due to their low temperature processing capability. The use of pocket is also described, in the context of, among other things, cutting instruments, in U.S. Pat. No. 6,494,882 by Lebouitz, et al., the disclosure of which is incorporated herein by reference.

As shown in FIGS. 3A, B, for multi-lumen catheters, access openings, according to certain embodiments, may be made only on outer wall 31 of the catheter 30 into a single lumen 32 in order to access electrical wires in that lumen without damaging other lumens 33, 34 of the catheter 30. FIGS. 3A, B illustrates such an opening 57, 58 as formed by, for example, a pulsed laser 60 ablation process.

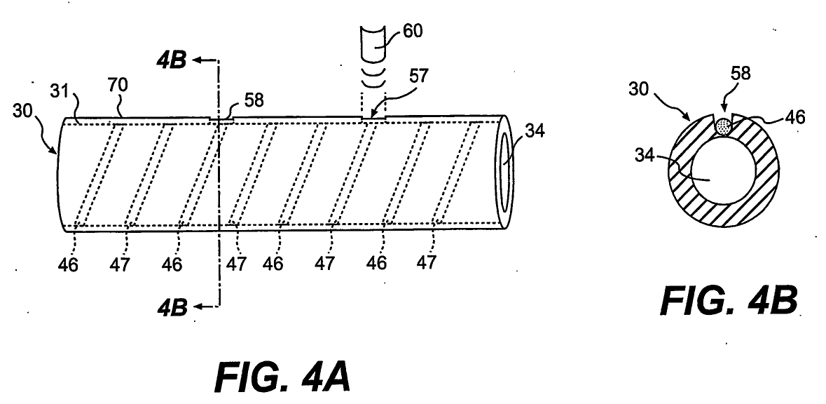

For catheters with embedded wires within their exterior wall, access openings may be made to expose a portion of wire without damaging the wire itself. For example, access openings may be made near the distal end of the catheter in order to connect one or more wires to a sensor material during a deposition process of the sensor layer. FIG. 4 illustrates access openings for a catheter that has 2 separate helically wound wires 46, 47 in its exterior wall 31. The exterior walls of a catheter are typically made from or coated with a conformal coating 70 of polymeric material. A low intensity UV laser 60, such as a 213 nm Nd:YAG laser, can be used to make access openings without damaging the metal wires underneath since most of the laser energy will be absorbed by polymeric catheter material due to the wavelength of the laser. By changing the type of laser, intensity, duty cycles, or any combinations thereof, access openings can be made on virtually any surgical instruments in a similar manner. For example, various ablation techniques using a pulse laser are described by Braren et al., “Laser ablation in material processing: Fundamentals and Applications”, Material Research Society, June 1993, the disclosure of which is incorporated herein by reference.

Etching or ablation to form access openings, pockets, or other relief features may be performed before or between any of the deposition processes explained below. Selective masking during wet or dry etching may also be used to form relief features on any layers. This may be accomplished by using a flexible shadow masking technique, as further explained herein, which can be used on both curved and flat substrate areas. Conventional photolithographic techniques also may be used on flat surfaces of surgical instruments, see, e.g., Mark Madou, “Fundamentals of microfabrication”, CRC Press, 2^{nd} Ed. (2002), the disclosure of which is incorporated herein by reference.

For certain applications, it may be desirable to deposit an adhesion layer in order to avoid or minimize delamination or peeling of a layer off of the substrate or other layers. Thus, as a third step includes the deposition of an adhesion layer to aid in the bonding among other layers or with the substrate. The adhesion layer may include, for example, Ti, Cr, TiW, epoxy, various polymer, parylene, or any combination thereof. The adhesion layer may also be a roughening of an existing layer.

Another step includes removal from the substrate of contaminants such as oils before performing depositions. This cleaning step may include the use of thermal methods (i.e., heating), chemical methods (ex., solvents), mechanical methods (ex., abrasion), or any combination thereof. Solvents include, for example, acetone, trichloroethylene, methanol, isopropyl alcohol, or any combination, which can be used to rinse and clean the substrate.

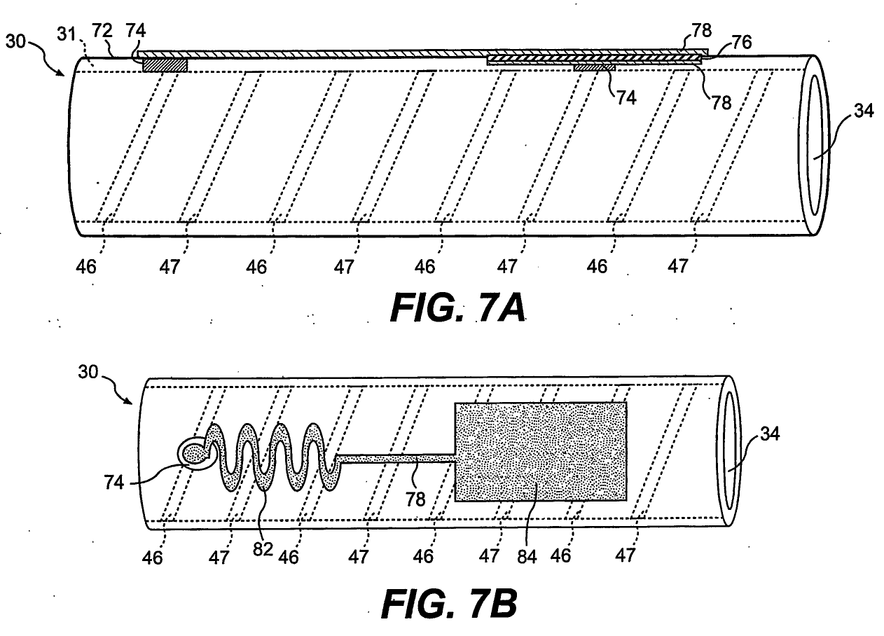

A fourth step involves deposition of the insulation, layer onto the substrate or other previously deposited layers. Deposition of layers such as an insulation layer can be achieved using a shadow mask to deposit material only through specific openings in the mask that define the desired feature. Additional details on the use of such masks, including a flexible shadow mask, are explained further below. FIG. 5 shows an insulation layer 72 on top of a catheter 30 that has 2 helically wound wires 46, 47 underneath the outer wall coating 70. Deposition of the insulation pattern is made to avoid, for example, unintentional filling of access openings made on the previous steps. For a typical catheter, the coating on outer wall is not electrically conductive. However, for conducting substrates or if wires are not completely covered either intentionally or non-intentionally by the outer wall coating, an insulation layer may be used as an electrical and thermal barrier between the outer wall and a layer above the insulation layer. In an alternative embodiment, this insulation layer may be deposited using a suitable dielectric material between two metal layers to create a capacitor, as shown in FIG. 7A, element 84 with metal 78-dielectric 76-metal 78 layers.

Insulation layers may be deposited through, for example, RF or pulsed DC sputtering of silicon dioxide, amorphous silicon, or silicon nitride. Numerous sputtering systems are available, including those provided by the Kurt J. Lesker Company of Clairton, Pa., such as PVD75. Silicon dioxide and silicon nitride can be sputtered both reactively or non-reactively with a heated or unheated substrate. The thickness of the insulating layer may depend on the type of material, the insulation requirements, and the type of the substrate, and may be on the order of, for example, thousands of Angstroms.

Plasma enhanced chemical vapor deposition (PECVD) may also be used to deposit layers such as an insulating layer. PECVD entails a chemical reaction of gases enhanced with plasma so that low temperature (under 400° C.) deposition of silicon dioxide, amorphous silicon, or silicon nitride is possible. A benefit of this process is that the resulting films are typically denser than those resulting from sputtering and may have better adhesion properties. A denser film may mean that there are fewer voids or pinholes that may allow subsequent layers to contact the substrate directly, causing possible shorts. However, PECVD is less directional and, therefore, there may be more deposition in areas not intended to have deposition. This problem can be addressed and overcome and compensated for by, for example, reducing the dimensions of the openings in the mask.

Silicon dioxide, amorphous silicon, or silicon nitride may also be deposited by low pressure chemical vapor deposition (LPCVD). LPCVD operates at temperatures at and below 400° C. for silicon dioxide and under 580° C. for amorphous silicon. This process is similar to PECVD except that the temperature is high enough that the chemical reaction continues on its own without the need for plasma. These films are denser than PECVD, but are even less directional.

Silicon dioxide may also be deposited by thermal evaporation. For example, an electron beam evaporator with water-cooled copper crucible is used to heat silicon dioxide disks to their evaporation point (1800-2000° C.). These evaporated films typically have lower packing densities, but lower intrinsic stresses than sputtered films.

Wet coating of silicon dioxide is yet another alternative deposition method. This method is typically a low temperature sol-gel process where the silicon dioxide is transitioned from a liquid or “sol” to a solid or “gel”. Sol gels can be deposited by wide variety of methods including dip coating, spraying, flow coating, spinning, capillary coating, silk screening, or rolling. Spray deposition of sol gels using an airbrush or automated spray system can be used and have been shown to deposit films of 100-220 nm in thickness. See, e.g., J. van Bommel, Glass Research, 7 (1997) 10-15, the disclosure of which is incorporated herein by reference.

The choice of insulation material and method of deposition will depend on the requirements imposed by the surgical instrument, intended medical application, and other layers. According to certain embodiments, if the substrate contains a low melting point material such as the coating on the outer wall of a catheter; Parylene coating can be very useful due to its room temperature deposition capability. Such coating is explained below in further detail in the protective coating step as it has good moisture and chemical resistant characteristics.

A fifth step is to deposit any necessary contact layers. A contact layer acts as an intermediate layer or a pathway among electrical wires, sensor layers, any other parts that requires an electrical connection. FIG. 6 shows one embodiment of a contact layer 74 deposited through access opening (see e.g., FIG. 5, openings 57, 58) for a catheter 30. The contact layer 74 provides a low resistance conductive trace between wires 46, 47 in the access openings and the sensor layer that is to be deposited over or in contact with the contact layer. A contact layer may be deposited, for example, using DC sputtering or evaporation of the required metal that will firmly stick to the wires or other layers underneath. As another example of forming a contact layer, conductive epoxy can be applied or injected though the access openings to fill the openings. As yet another example, electroplating or electroless plating of metal onto the conductive trace through the access hole may be used to form a contact layer. Electroplating of a metal on another metal (for example, nickel, copper, or chromium electroplating on exposed conductive traces as presented here) is a well-known technique in the art of micro fabrication. Fundamentals and techniques can be found in Paunovic, M. and Schiesiner, M., “Fundamentals of Electrochemical Deposition”, John Wiley and Sons, New York, 1998 and Dennis, J. K. and Such, T. E., “Nickel and Chromium Plating”, Butterworths, 2^{nd} Edition, 1986, the disclosures of which are incorporated herein by reference. A still further example entails using metal sputtering or evaporation. Numerous metal sputtering or evaporation systems that are capable of this deposition exist, such as those provided by the Kurt J. Lesker Company of Clairton, Pa., such as PVD75. The metal target, which can be, for example, aluminum, copper, gold, silver, platinum, or any other sputterable or vaporizable conductor, is also available from numerous vendors including the Kurt J. Lesker Company of Clairton, Pa.

According to certain embodiments, a portion of the contact layer may be large enough to make appropriate contact with wires or cables. In an alternative embodiment, the contact layer can be deposited as a part of wiring such that two or more sensors are connected over the substrate (i.e., the exterior wall of the catheter). These traces can be extended outside of the access opening or pocket and along the tool to a suitable attachment point in order to connect a sensor with other sensors, measurement devices, and power generators. FIG. 7A,B illustrates this point. Contact layers 74 are used as a wire or inter-connect between the two sensors 82, 84. A capacitive element 84 may be created by depositing a suitable dielectric 76 between two metal layers 78 as shown in the figure. This example shows that capacitive 84 and a resistive 82 sensor connected in series by the metal layer 78. A sensor can be formed on a pocket (unlabelled recess containing stacked layers 78, 76, 78) as depicted in FIG. 7A in order to maintain uniform topography of the layers. In yet another embodiment, contact layers can be deposited simultaneously with sensor layers if the material is suitable as a sensor material as well as a conductive trace. Material of this layer can be the same as sensor layer or be different as long as, functionally, it allows necessary electrical and/or thermal conduction between sensors and wires. The number of deposition and size for the contact layers are varied based on geometry of access openings and other layers.

A sixth processing step according to the present invention is to deposit the one or more sensor materials. Depositions of the one or more sensor materials (and other layers such as insulation, contact, and encapsulation layers) can be realized by various physical and/or chemical deposition techniques that are known in the art of micro fabrication, including, for example, sputtering, thermal evaporation, PECVD, LPCVD, MOCVD, and Pulsed Laser Deposition (PLD). Various CVD techniques are described by Pierson, Hugh O., “Handbook of Chemical Vapor Deposition”, Noyes Pub., 2^{nd} Ed. 1999, the disclosure of which is incorporated herein by reference. Various PDV techniques are described by Mattox, Donald M., “Handbook of Physical Vapor Deposition: PVD processing”, Noyes pub. 1998, the disclosure of which is incorporated herein by reference. Relevant PLD techniques are described by Douglas B. Chrisey, Graham K. Hubler, “Pulsed Laser Deposition of Thin Films”, John Wiley and Sons, 1994, the disclosure of which is incorporated herein by reference. According to certain embodiments, an important factor in choosing and using one of these deposition techniques is how local temperature affects the substrate and other previously deposited layers during the deposition processes. Deposition parameters of such processes have to be also adjusted so that local temperature on a substrate and previously deposited layers do not reach an unacceptable level.

Deposition method further include, for example, (a) laser deposition techniques, where traces or features can be formed by techniques commonly understood by one skilled in the art of laser-based material deposition, whereby the trace(s) or features can be patterned onto or into the surface through the physical interaction of a laser and a source material(s) appropriate to the trace composition, with the pattern and geometry of the deposited trace(s) or feature being defined by a design specification or database, such as one derived from a computer aided design (CAD) tool; (b) ion beam deposition or implantation techniques, where trace or features may be formed by using ion-beam material deposition, whereby the trace(s) or features can be patterned onto or into the surface by an ion beam, which may be used alone or in a physical/chemical reaction with another material source(s), with the pattern and geometry of the deposited trace(s) being defined by a design specification or database; and (c) damascene techniques, where trenches or grooves are created in the surface of the instrument corresponding to the nominal pattern of the trace(s), and this groove(s) is filled with the conductive material(s) of choice by electroplating or other methods to provide conductive material into the groove(s). Still further, consistent with certain embodiments embodiment, sensors may layers may also be fabricated using stamping, wicking, or molding, that, functionally, can provide the desired patterning onto the substrate, be it flat, curved, or a thin conformal film.

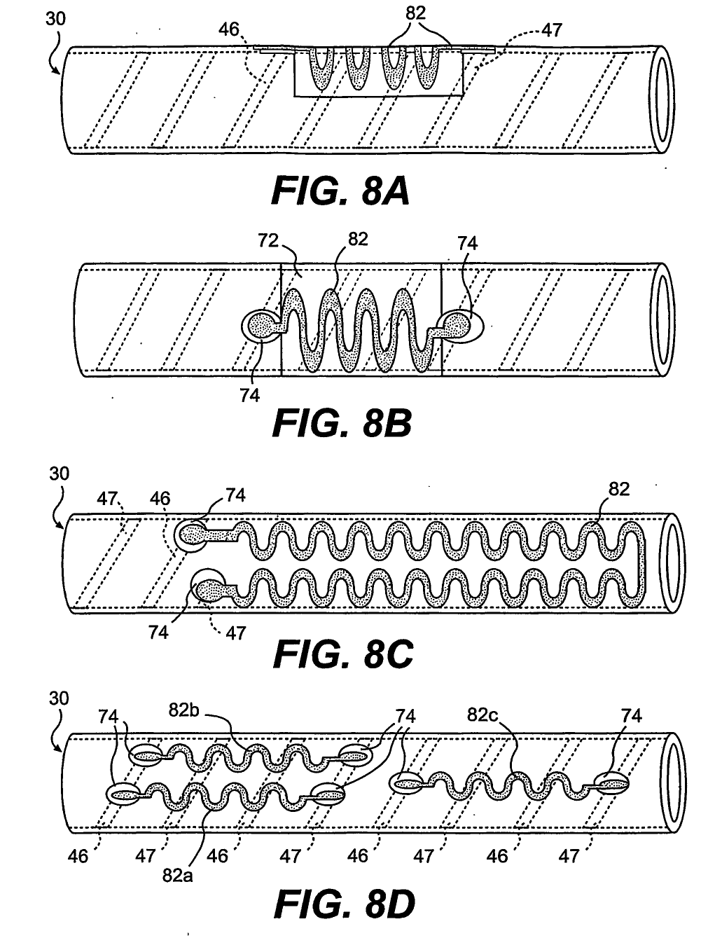

FIGS. 8A, B shows one embodiment of a blood velocity sensor 82 that is deposited after the five aforementioned steps on a small portion of catheter 30. In this embodiment, a low power sputtering technique can be used to limit the substrate temperature to below 60° C. Deposition thickness and geometry of the sensor 82 are adjusted based on its required resistance, heat generation, and heat distribution characteristics. FIG. 8C shows another embodiment of a catheter 30 with a sensor 82 that is relatively larger and of a different geometry than in FIG. 8A,B. Series (ex., 82A and 82C) or parallel (82A and 82B) combinations of resistive sensors can be realized as shown in FIG. 8D. Sensor 82 can be also combined with capacitive sensors 84 shown in FIG. 7 in order to realize a variety of RC configurations.

According to one embodiment of a blood velocity sensor according to the present invention, a sensor layer contains one or more thermoresistive materials. Selection of an appropriate thermoresistive material may be based on consideration of the thermoresistivity of the material as well as processing required for the deposition. According to certain embodiments, the sensor materials for blood velocity sensor applications have thermoresistivities of greater than +/100 ppm per degree Celcius. Such materials include, for example, silicon, gold, nickel, iron, tungsten, platinum, copper, silver, aluminum, chromium, their alloys and composite materials with ceramics and various metal oxides of nickel, cobalt, copper, iron, vanadium and manganese. Typically, metal films have a positive thermoresistive coefficient such that their resistance values increases with an increasing temperature. Their thermoresistive response is typically linear or approximately linear over a relatively large temperature range. On the other hand, non-metal films generally have a negative thermoresistive coefficient and generally have non-linear thermoresistive responses, unlike the metal films. However, they typically have higher temperature coefficients of resistance compared to that of metals which can be beneficial to, essentially, amplify small thermal changes into large resistance changes. See generally “Thin Film Thermistors” by Morris et al., “Thin film thermistors”, Journal of Physics Engineering, Scientific Instruments (1975) Vol. 8, pp. 411-414, the disclosure of which is incorporated herein by reference.

A seventh step is to enclose with a protective layer any parts and layers that need to be protected from operating environment of the sensor. FIGS. 9A, B shows a part of catheter 30 that has a sensor 82 formed on its exterior wall covered with a protective coating 79. The protective layer may be an electrically nonconductive, biocompatible material that is very conformal to cover any irregularity of a layer underneath. For a blood velocity sensor according to the present invention a protective layer may consist of a blood-compatible coating of a few hundreds or thousands of nanometers that is thick enough to provide protection and thin enough to allow heat transfer between the sensor layer and the blood stream. Example of such coating are PARYLENE-N® or C®. Typically, PARYLENE-N® coating is very conformal. It also has a very low coefficient of friction, which is desirable in many surgical applications that involve contact with bodily fluids and tissues. PARYLENE-C® has similar properties of PARYLENE-N®, but also possesses superior moisture protection capability. These parylene coatings can be applied using, for example, a parylene coater developed by Specialty Coating Systems, IN., such as PDS2010. See generally Satas et al, “Coating Technology Handbook”, 2^{nd} Ed. CHIPS (2000), the disclosure of which is incorporated herein by reference. Other materials such as glass, quartz, carbon, and moisture-resistant dielectrics may be used alternatively or additionally.

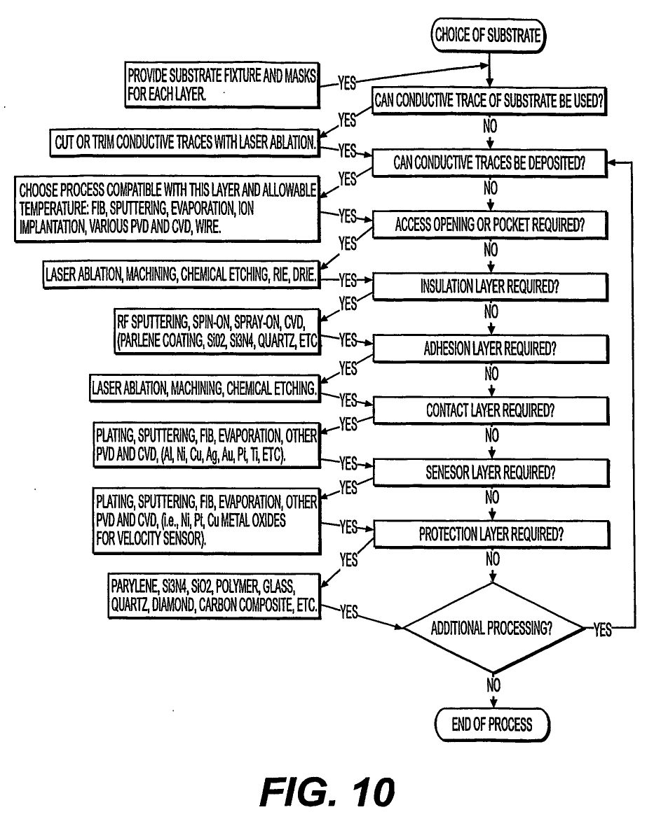

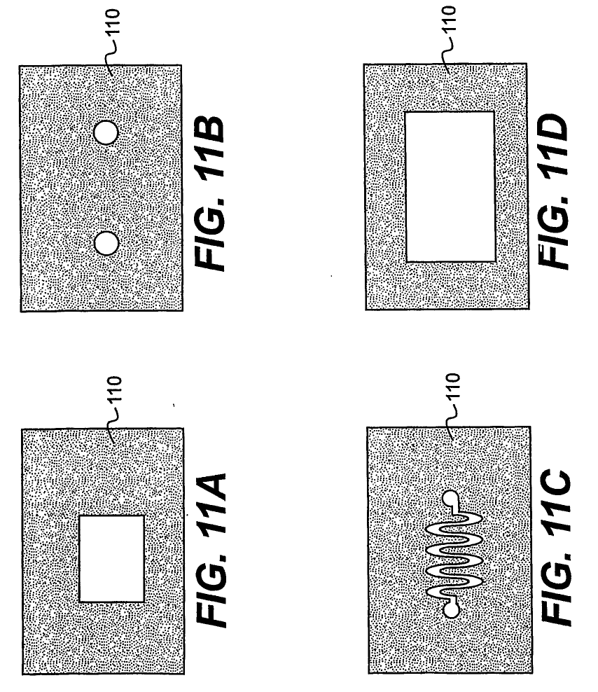

A flow chart of exemplary processing steps according to certain embodiments of the present invention is shown in FIG. 10.

As illustrated by the presented basic steps of making a sensor on a catheter, the same fabrication processes and preferred method of sensor formation can be applied to form various sensors on other curved and flat surgical instruments as well. According to certain embodiments, sensor formation uses flexible shadow masking, laser deposition, 3-D thin-film deposition or combinations thereof.

Flexible shadow masking uses flexible masks to at least partially conformally cover and selectively expose certain areas of curved surfaces during thin-film deposition, and may also be used with flat surfaces as well. Varying mask thickness and material type, including thin metal and polymer sheets, may be used. Functionally, the mask should be flexible enough to at least partially follow the contour of the substrate, such as the curved contour of a catheter. The patterns in the shadow mask are made using, for example, chemical etching, electroforming, and/or laser ablation. The mask thickness may range, for example, from sub-micron to hundreds of microns. Minimum size of a pattern that can be deposited through a mask opening reduces with the increase of mask thickness. Materials suitable for the mask include, for example, stainless steel, brass, copper, Mylar® and Kapton®. During any thin-film deposition process mentioned above, the shadow mask is used to allow deposition of material only on the desired portions of the substrate or any layers through the intentional pattern openings made on the mask. FIGS. 11A-D shows such masks 110 used for the presented deposition steps of insulation layer (FIG. 11A), contact layer (FIG. 11B), sensor layer (FIG. 11C), and protection layer (FIG. 11D) that are depicted in FIGS. 5, 6, 8, and 9, respectively.

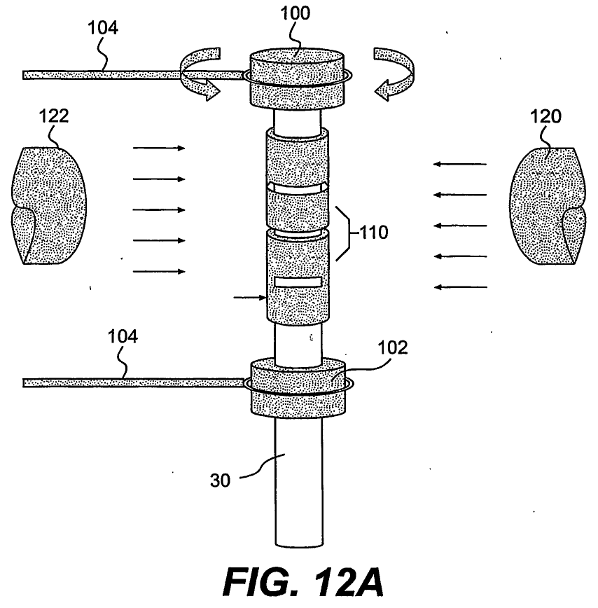

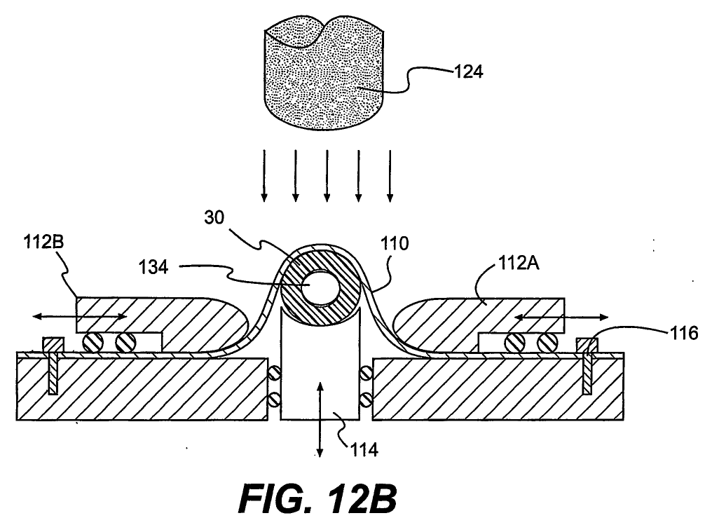

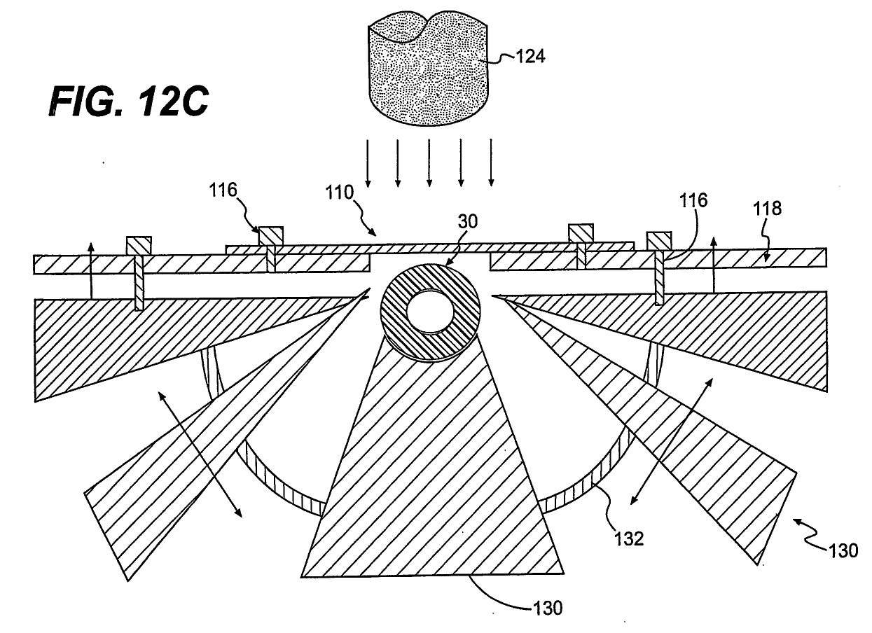

During a thin film processing, one or more masks and a one or more substrates may be held in a specially designed fixture to hold them and control their location and exposure. A substrate fixture can be made to move and rotate relative to the substrate as needed such that uniformity of deposition can be controlled over non-flat surfaces of a substrate. The substrate fixture may include the ability to mechanically clamp and align a mask to a desired location relative to a substrate and other layers. FIGS. 12A-C illustrate various embodiments of substrate fixture and mask arrangements.

FIG. 12A shows a contact flexible mask 110 that is wrapped around a catheter 30 which rotates with end grippers 104, 100 and 104, 102 to expose desired mask openings all around the catheter. The rotation angle and speed are adjusted to create desired deposition from one or more deposition sources 120, 122 through the mask 110.

FIG. 12B illustrates a contact, flexible mask 110 that is used to partially follow the curved surface of the substrate 30. The substrate can be internally supported by, for example, a guidewire or the like 134. Additionally, several flexible masks can be aligned and stacked to generate multiple patterns. The relative alignment and bending of the mask 110 relative to the substrate 30 can be achieved using positioning elements 112A,B, 114. The mask may be held in place and aligned with alignment holes 116. A deposition source 124 provides material to be deposited through holes in the mask 110 onto the substrate 30.

FIG. 12C shows a substrate fixture that holds and controls a non-contact, flexible shadow mask 110 with respect to the substrate 30. As depicted, the shadow mask 110 can be aligned using alignment holes 116 and a mask holder 118, connected to a substrate fixture 130. The substrate fixture may be opened or closed along the helical thread 132 to adjust the bending of the mask 110, which is shown in an unbent position but which may be curved to better follow the curved profile of the substrate 30. The small allowable distance between the mask 110 and substrate 30 increases with the minimum size of pattern openings in the mask 110. In a certain embodiment, this non contact shadow mask may be used to avoid contact between the mask and the layer underneath.

In certain embodiments, etching processes, such as chemical wet etching, Reactive Ion Etching (RIE), Deep Reactive Ion Etching (DRIE), may be used to make the access openings or pockets using a flexible shadow mask as an etching protection layer. As shown in FIG. 12A, such masks 110 may have a pre-deposited adhesive layer on one side, which can be made out of thin Teflon, MYLAR® or KAPTON® tape to attach the mask 110 to the substrate 30. If such flexible shadow mask is affixed to cover a curved surface, the openings in the mask permit etching chemicals to attack the material underneath the opening, thereby allowing etchings on curved surfaces. Etching resolution may be low compared to a conventional etching technique using photoresist as a chemical protection layer, which is only effective on a flat surface. However, this etching technique using temporarily affixable, flexible shadow mask is useful for making access opening, pockets, patterns on non-flat substrates and layers, and could also be utilized on flat surfaces, if desired. After necessary etching is done the affixed mask may be removed, for example, by peeling off.

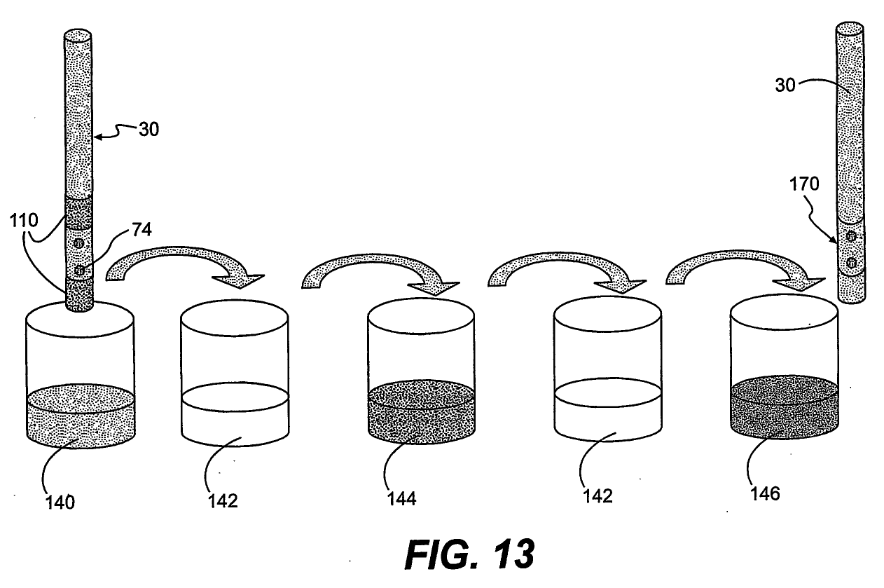

Apart from the aforementioned deposition techniques that require vacuum equipments, plating, such as, electroless plating, can also be utilized to deposit the necessary metal layers (i.e., adhesion, contact, or sensor layers) directly on a surgical instrument with or without a contact flexible mask. Electroless plating is a chemical process of depositing a thin metal. For one exemplary embodiment of the blood velocity sensor, a thermoresistive material such as nickel can be deposited as the sensor layer using the electroless plating. FIG. 13 describes a basic example of an electroless nickel plating process on a catheter. By using affixable flexible masks 110, nickel as a sensor layer 170 can be deposited only on a desired portion of the catheter. As depicted, the substrate 30 with mask 110 is first (after optional pre-processing steps such as one or more of cleaning and depositing one or more layers, such as insulation and contact layers) introduced to a stannous chloride, hydrochloric acid solution 140, water 142, and a palladium chloride, hydrochloric acid solution 144, to precipitate palladium on the surface at locations exposed by the mask 110. The precipitated palladium will serve as a catalyst for the electroless deposition of the nickel. After rinsing in water 142, the masked substrate is then introduced to a nickel sulfate, sodium hypophosphite, sodium succinate, succinic acid solution, where the nickel is deposited. All the above chemicals can be purchased from suppliers such as Fisher Scientific, and Technic. Other electroless plating materials and techniques may also be used, the present illustration merely being one example. Electroless plating is further described by Paunovic, M. and Schiesiner, M., “Fundamentals of Electrochemical Deposition”, John Wiley and Sons, New York (1998), the disclosures of which is incorporated herein by reference. The application of electroless plating of sensor materials on a surgical instrument can offer an economical way to realize volume manufacturing of various sensors.

In yet another embodiment, as an alternative method of depositing a sensor layer, as shown in FIGS. 14A, B a thin polymer such as MYLAR®, KAPTON®, PI® can be used as an additional substrate 172. Again, similar to the affixable mask, this polymer, with a thickness of, for example, less than 50 microns, has adhesive on one side, which acts as a thin tape. On the other side, a sensor layer can be deposited by any suitable deposition techniques that are presented. Once the sensor layer or layers are fabricated on the additional substrate, the thin substrate 172 is attached to the surgical instrument 30. A pocket or access opening 150 can be formed on a surgical instrument 30 such that it can contain the substrate 172 to compensate for the increase of overall thickness due to the additional substrate. In order to connect a sensor on the flexible substrate 172 to the contact layer 74, an additional contact layer or a metal layer 78 can be formed between them using the presented shadow mask technique after the attachment of the flexible substrate onto the catheter. Again, a protective layer can be subsequently deposited to cover them. This alternative method can also offer a cost effective way to produce a large number of sensors.

The fabrication techniques according to the present invention can be used to create a variety of thin-film sensors on flat and non-flat surfaces of other medical or surgical instrument in addition to the exemplified catheter. For example, the exemplary sensor fabrication processes on a catheter can be implemented with other sensors having application in detection of distance, pressure, temperature, density, and the presence of nerves. Examples of surgical tools on which such sensors can be formed include, but are not limited to, ablation tips, needles, blades, probes, cannula, forceps, grippers, micro-grippers, endoscopic tools, and other end-effectors of surgical instruments. Examples of material types for these substrates include plastics, stainless steel, titanium, and carbon steel. The choice of thin film layers, their processing order, and the deposition techniques will be adjusted according to the temperature and other requirements imposed by these surgical tools, the desired sensor type or types, and the intended application or use of the device.

In one embodiment, this directly deposited, conformal, thin-film blood velocity sensor includes a thermoresistor (RTD or thermistor), which operates on the basis of a change in its resistance with respect to a change in its temperature. A thermoresistor may be used as either a heating element or a temperature sensing element, and configuring a given thermoresistor for one type of operation (e.g., heating) does not bar it from another type of operation (e.g., temperature sensing), at the same time or a different time.

The operation of the thermoresistor blood velocity sensor starts with heating of the sensor layer by passing a current through it. The sensor generates heat according to Joule heating. As known in the art of thermal anemometry, such thermoresistive material, either as a RTD or a thermistor, in conjunction with generation of heat can be used to measure flow velocity of gases or liquids. The application of this basic idea to a medical device has been described by Delaunois et al. in early 1970s and, according to certain embodiments, the present invention offers a new practical method of realizing such ideas by forming integrated sensors directly on surgical instruments. See, Delaunois et al., “Thermal method for continuous blood velocity measurement in large blood vessels and cardiac-output determination”, Medical and Biological Engineering (1973) Vol. 11, No. 2, pp. 201-205, the disclosure of which is incorporated herein by reference.

For instance, according to certain embodiments, a conformal blood flow sensor at the surface of a catheter can be formed without manual handling during the sensor integration with the catheter as seen from the presented examples. Moreover, overall increase of catheter diameter can be negligible due to relatively thin geometry of the sensor. As illustrated in FIGS. 14A,B, the use of a pocket can also eliminate any increase in diameter that may be caused by multiple depositions of several layers. As the sensor is directly deposited over the surface of the catheter, a wide variety of designs are possible. Features can be deposited virtually anywhere on the surface of the catheter with various sizes and orientations. The sensor can occupy entire section of a catheter or covers only a small portion of a catheter. The sensors in different numbers, sizes and orientations will be chosen based on the requirement of a particular application considering its reliability and sensitivity.

For the operation of sensor, there are many different anemometry scheme such as constant current anemometry (CCA), constant temperature anemometry (CTA), constant voltage anemometry (CVA), constant heat flux anemometry, and other approaches in which the variation of the electrical signal in the sensor is correlated to flow. Fabrication methods according to the present invention can be used to prepare regardless of the type of operation scheme to be employed. The CTA technique, for example, utilizes a feedback amplifier to maintain the average sensor temperature and resistance constant, within the capability of the amplifier. The practical frequency or speed to detect fluctuations of flow using CTA is thus limited by the frequency at which the feedback amplifier becomes unstable. A feedback circuit drives the sensor to the desired temperature, such as less than 10 degrees above the body temperature, and maintains it. In order to maintain a reasonable dynamic range, it may be desirable to rapidly reach the desired temperature without too much overshoot after a temperature perturbation due to the changes in blood flow. More descriptions are found in Hardy et al., “Flow Measurement Method and Applications”, John Wiley and Sons (1999), the disclosure of which is incorporated herein by reference.

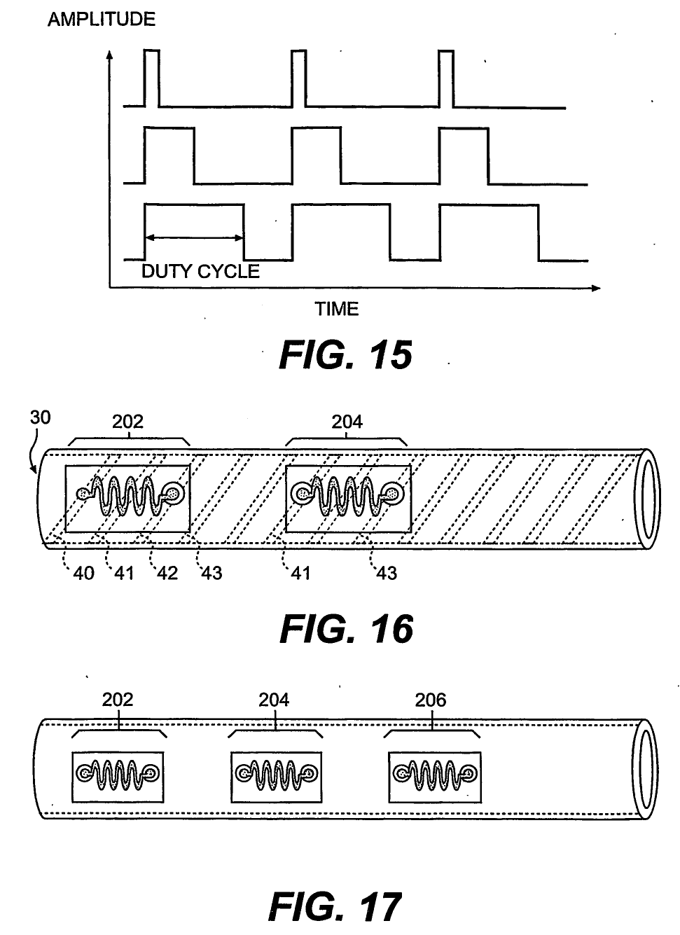

In an alternative embodiment, blood velocity sensors can be operated in a digital mode with modulated input current. Pulse-Width-Modulation (PWM) anemometry is one such technique, which can be also applied to the blood velocity sensor operation on catheter. The application of a PWM technique can increase resolution of the sensor. Flow sensors can be heated from PWM signals as shown by the three exemplary waveforms in FIG. 15, the sensors cooling response due to blood flow changes the duty cycle that is required to maintain the sensor at a constant temperature. The faster the flow is, the faster the cooling becomes and the longer the duty cycle of the pulse is required for maintaining the sensor at a constant temperature. For a fixed duty cycle, the amplitude of the pulse may alternately be adjusted to maintain a constant temperature, such as increasing the amplitude in response to increasing flow rates. The heating magnitude is thus controlled by adjusting the amplitude, duty cycle, or both of the input pulse. The frequency of the pulse can be, for example, in the range of from hundreds of hertz to thousands of hertz in order to produce uniform heating throughout the sensor layer. More descriptions can be found in Foss et al., “The Pulse Width Modulated Constant Temperature Anemometer”, Meas. Sci. and Tech. (1996) Vol. 7, pp. 1388-1395, the disclosure of which is incorporated herein by reference.

Multiple arrangements of sensors can be useful for different operating situations. For example, in one embodiment for the application of blood velocity sensing, a single sensor, fabricated according to the present invention, can be used. A change in blood flow rate varies the convection rate on the heated sensor surface, which in turn changes the temperature of the sensor and its resistance. Thus, either CCA or CTA can be used to detect the change and generate to flow rate information. This configuration measures a blood flow rate but not the flow direction.

In another embodiment of this invention, as shown in FIG. 16, in the application of blood velocity sensor, two thermoresistors 202, 204 (i.e., two thermal-resistive heating elements) are used. In this configuration, more than two separate wires 40, 41, 42, 43 may be needed to operate the sensors separately. By periodically switching the heating between two thermoresistors, one thermoresistor can be used as a temperature sensor while the other is being used as a heater. Flow rate is sensed in the same way as when a heating thermoresistor operates as the single sensor configuration. Flow direction is sensed by an unheated thermoresistor while the other thermoresistor is being heated as the blood flow carries some of the generated heat. The unheated thermoresistor senses an increase in temperature if the blood flows from the heated sensor to the unheated one. By periodically switching the heating between two thermoresistors, the direction of blood flow can be intermittently monitored.

In yet another embodiment, as shown in FIG. 17, three or more sensors 202, 204, 206 can be used. Each sensor can be connected to, for example, a pair of embedded wires (not shown). This configuration also allows the sensors to measure both flow rate and direction. In this configuration one thermoresistor, ex. 204, can act as a heater while the others 202, 206 act as temperature sensors. Again, flow rate will be sensed by the heating thermoresistor and different temperature response between two temperature sensors is an indicative of flow direction. The sensor located in the direction of flow with respect to the heater will experience a slightly higher temperature as the flow carries the heat over. Size of the heating thermoresistor and sensing thermoresistors can be optimized for a particular sensitivity.

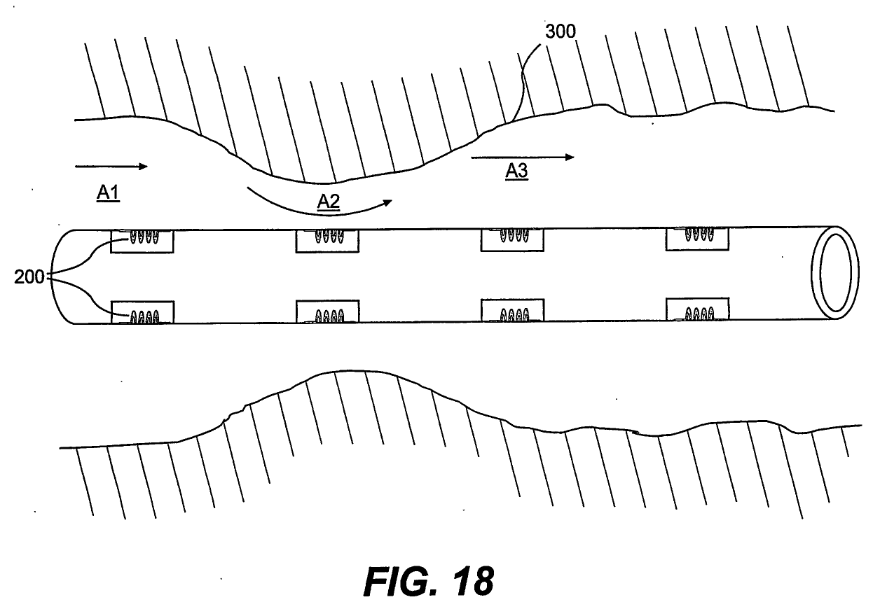

These exemplary embodiments of conformal thin-film blood velocity sensors, fabricated according to the presented invention, offer certain benefits. Due to their minimal size and thickness, the system response time is relatively fast and measurement is localized. This allows potentially faster and more accurate measurement of blood parameters at a given location within the blood vessel, which is often desirable in the surgical environment. Global measurement is also possible by using multiple sensors that are appropriately spaced throughout the body of catheter. FIG. 18 shows such multiple sensors 200 on a catheter which can be used to detect the degrees of a partial blockage, e.g., area A2, due to plaque within the blood vessel 300. The flow rate sensing around a blockage A2 will yield a higher value than the adjacent areas A1, A3. The degree of flow rate difference can be related to the degree of the blockage. Also, by using ultrasonic sensors, which are explained below, in conjunction with the flow rate sensors, one can quantify a degree of the blockage.

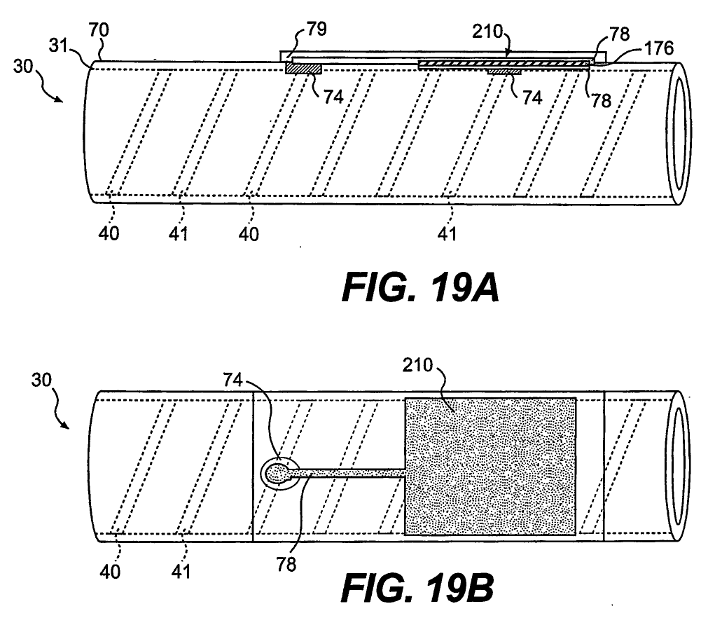

In addition to the blood velocity sensors on a surgical instrument, as illustrated in FIG. 19A,B, an ultrasonic transducer 210 can be also formed by integrating a piezoelectric layer 176. The ultrasonic sensor layer 176 is deposited with suitable piezoelectric material such as ZnO, PZT, or PVDF. The sensor layer can be deposited directly on a surgical instrument as the substrate or be deposited on an additional substrate and be affixed into a pocket made on a surgical instrument. Through the use of, for example, a pocket and metal layers, a piezoelectric film can be sandwiched between the two metal electrodes. Each electrode is connected to a different wire 40, 41 such that an input voltage potential can be applied across the thickness of the piezoelectric film for generating ultrasonic waves.

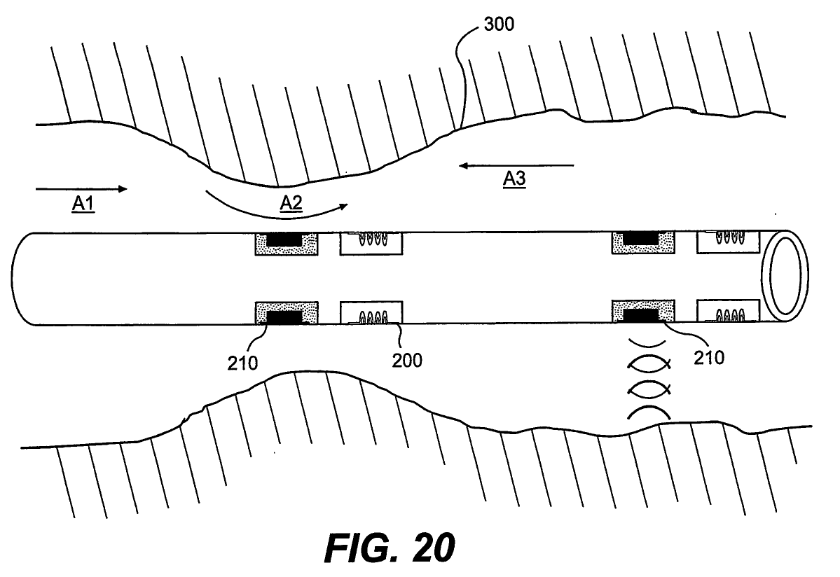

A piezoelectric film excited with alternating voltage input can generate ultrasonic waves through the medium which it operates. As the generated wave propagates through a medium (e.g., blood), the wave will partially reflect back at the medium of different density (e.g., blood vessel walls or plaque). The propagation speed of such ultrasonic waves in a desired medium (e.g., blood) is known or can be determined through a calibration. Thus, using a control circuitry, when the duration between the time of a pulse generation and that of the pulse reflection off of another medium (e.g., blood vessel) is obtained, the distance between the ultrasonic transducer and the reflection medium are calculated. This is known as a pulse-echo operation of ultrasonic transducer. Finding the relative distance enables the estimation of blood vessel geometry. As shown in FIG. 20, multiple ultrasonic transducers 210 can be used to increase the range and accuracy of measured dimension. Blood pressure within a certain part of vascular system, where certain flow assumptions (such as laminar, steady, incompressible, and uniform flow) become valid after a proper calibration, can be estimated by measuring a blood flow rate and a cross-sectional area of blood vessel. Using ultrasonic transducers and flow sensors together, one can measure a blood pressure at a given point within the vascular system. Simultaneous detection of blood pressure, blood flow, and vessel blockage is then possible with the distributed flow sensors 200 and ultrasonic sensors 210 as also shown in FIG. 20.

In another embodiment, a strain sensor can be also realized on a surgical instrument in the similar manner. It can be either directly deposited on a surgical instrument or on a separate substrate then affixed to the surgical instrument. Again, a sensor layer of the presented method is formed with a suitable strain gage material such as silicon and its variations while other processes of the present invention stay the same. The resistance of such material changes as it is stretched or compressed and by measuring the change in resistance, a local strain can be calculated. Local bending, twisting, and stretching can be extracted by using different orientations of strain sensors with respect to the surgical instrument.

It will become apparent that any combination of the presented sensors can be formed using the presented method of integration on the same catheter or the same surgical instrument.

An electrical drive circuit and an electrical sense circuit can be coupled to the sensors according to the presently claimed invention. According to one embodiment, a single circuit may be developed to provide both functions. Alternatively, more than one circuit may be used to provide these functions.

An electrical drive circuit can be designed to, for example, apply the correct amount of current to a sensor. A function of the electrical drive circuit can be to apply the correct voltage to a thermoresistive element to provide sufficient current to resistively heat the element to, for example, just 1° C. above the temperature of the blood. Thus, the circuit can measure electrical resistance and adjust drive voltage to maintain electrical resistance at a set point.

The electrical sense circuit may also measure the current flowing through, for example, a thermoresistive element. For example, the output of a thermoresistive element may be determined by measuring the electrical current flowing through it, thereby inferring the amount of thermal heat being absorbed by the surrounding blood. A knowledge of the magnitude of the current can correlated with, for example, blood velocity, and thus the electrical sense circuit can be used to determine, for example, the conditions of either flow or no-flow of blood, or a relative flow rate.

### EXAMPLE 1

Conductive Traces on Substrate

A Renegade® HiFlo catheter (Boston Scientific, Inc.) was used to illustrate an embodiment of the direct integration of a conformal micro-fabricated sensor onto a catheter. The size of Renegade® HiFlo catheter is 3 French, which means its nominal outer diameter is about 0.99 mm. The catheter contains embedded wiring, as generally shown in FIG. 1C. Specifically, the catheter contains two separate helically wound wires, which are visible through a thin, semi-transparent, polymeric exterior wall of the catheter. The exterior surface of the catheter served as the substrate for a micro-fabricated conformal sensor and the two embedded wires within the exterior wall of the catheter served as the conductive traces for electrical signal.

Insulation Layer

The substrate (the exterior wall of Renegade® HiFlo catheter) is a relatively low melting point polymer material. Parylene was chosen as an insulation and adhesion layer over the catheter, since it can be deposited at room temperature (˜25° C.) avoiding any possible melting of the substrate. The Parylene was coated over the distal end portion of the catheter using a Parylene deposition system (Model 2010 LABCOTER, SCS, Inc). Parylene dimer C is a powder form of Parylene that is consumed by the Parylene deposition system. Approximately 8 grams of the dimer were used to deposit approximately 5 micron thick Parylene coating onto the catheter. The catheter, except for the distal end of the catheter (˜2 cm), was covered during the deposition process to prevent unwanted deposition. The Parylene coating was thus deposited only on the exposed portion of the exterior walls at the distal end up to 2 cm.

When visually inspected, the Parylene coating was almost undistinguishable from the original catheter itself since it is a clear thin coating.

Access Openings and Pockets

Holes were made into the catheter outer body using a UP213 laser ablation system (NEWWAVE, Inc.), which emits 213 nm wavelength of Nd:Yag UV laser. The UP213 laser ablation system is an in-situ laser ablation machine that allows visual inspection of a hole being made during the actual ablation. For one hole (an access opening), the Parylene coating and the exterior wall above the first wire were burned off just to expose the first wire undemeath. An additional access opening was also made to expose a second wire.

Another hole formed in the outer body was a pocket (recess).

Contact Layer

To form contact layers connected to the exposed wires in the access openings, nickel was deposited into the access openings through the hole using a mask, and a conductive epoxy was applied. The mask, as shown in FIG. 11B, covered all but the two holes thus allowing the deposition of nickel through the openings, as discussed further below with respect to the masks and their alignment. The end result after this step is generally depicted as in FIG. 6, but with differences in the area of insulation layer (Parylene).

Nickel was deposited using a DC sputtering machine with the following condition: 40 mT, 200 W, for a few hours in order to deposit a few microns. Conductive silver epoxy (MG industry, Inc. 8331 14G) was then applied to fill up the holes up to the level of the outer surface of the catheter. Nickel and conductive epoxy have also been used individually to form the contact layer.

Sensor Deposition

Using a CAD program, Solidworks (Solidworks Corporation, Concord, Mass., mask designs were made. The design files were sent to a third party vendor, Gateway Laser Service, Inc. (St. Louis, Mo.), to make a patterned opening on flexible 75 micron thick brass sheets according to the mask design. Then, the masks were aligned under a microscope relative to the catheter substrate in preparation for sensor deposition.

A brass mask with a serpentine patterned opening, as generally shown in FIG. 11C, was used to form the sensor layer. The pattern of a nickel sensor layer was deposited through the mask opening using the DC sputtering system (Model SC2000, Vacuum Process Technologies, Plymouth Mass.) with processing conditions of 40 mT, 200 W, and 45 min to yield a nickel layer with an estimated thickness of 0.2˜0.3 microns and the structure generally shown in FIG. 8A, B.

Protection Layer

Parylene coating protection layer having the same approximate thickness as the Parylene insulation layer was deposited using the same process condition and machine mentioned in the insulation step.

### EXAMPLE 2

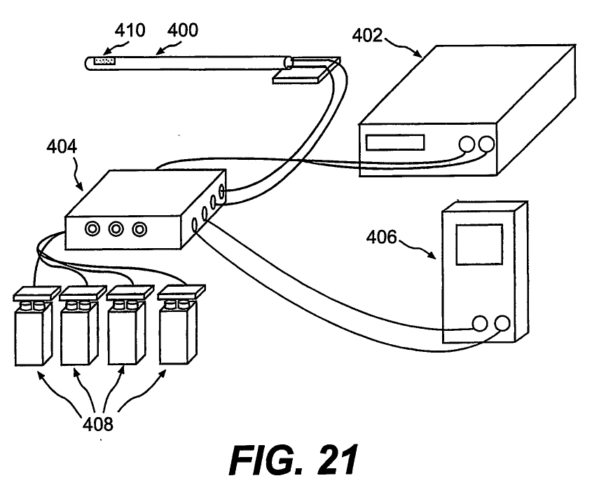

A sensor control and test system that was used to characterize sensor responses is schematically shown in FIG. 21. The system consisted of a power supply 402, control circuit 404, output monitor 406, and batteries 408. The sensor 410 on the catheter 400 was connected to the control circuit 404. Testing was done using this system to characterize the response of a catheter flow sensor to fluid flow. A catheter 400 having a sensor 410 according to Example 1 on its distal end was inserted into a tube slightly bigger than the catheter itself to mimic blood vessel. The tube contained either a glycerin-DI water solution, which is a semi-viscous solution commonly used to model blood flow, or DL-water. Solution flow was controlled by pump connected to the tube end opposite where the catheter was inserted.

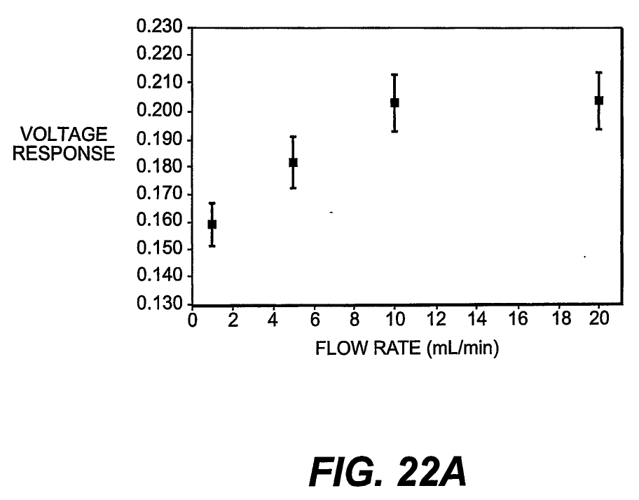

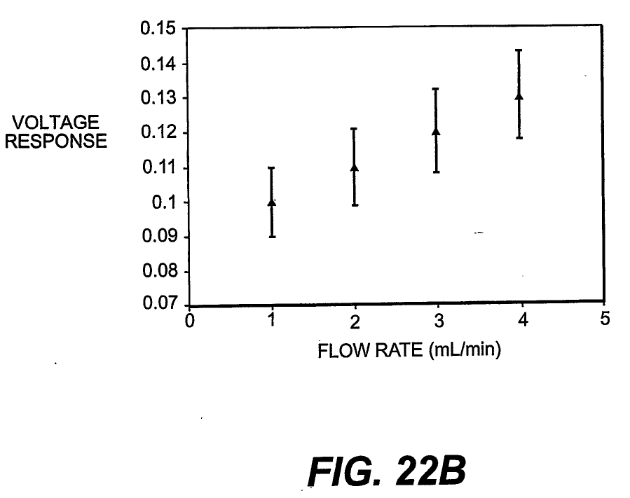

The sensor response, operating in constant current anemometry (CCA) mode, in a glycerin-DI solution (40:60) using a syringe pump are shown in FIG. 22A. Mean sensor response values (mL/min) are shown (points), with error bars indicating plus and minus fluctuation ranges attributed to the flow rate control tolerances.

The sensor response, operating in constant current anemometry (CCA) mode, in DI-water using a peristaltic pump in the same configuration are show in FIG. 22B. Mean sensor response values (mL/min) are shown (points), with error bars indicating plus and minus fluctuation ranges attributed to the flow rate control tolerances.

### EXAMPLE 3

A temperature sensor was integrated onto the surface of a RF ablation tip (Johnson & Johnson's). Accurate and fast temperature monitoring of an ablation tip surface is importance since it is the best indication of how well the RF ablation of a tissue is performed. Conventionally, the temperature of the RF ablation tip is measured with a thermocouple embedded inside the tip, but not at the surface. A sensor was deposited on the RF ablation tip, which is a cylindrical shaped metal substrate approximately 3 mm in diameter and 8 mm in length, generally according to Example 1. The location of the sensor (width 0.5 mm×length 7 mm×thickness 0.5 microns) was on the side wall of the ablation tip.

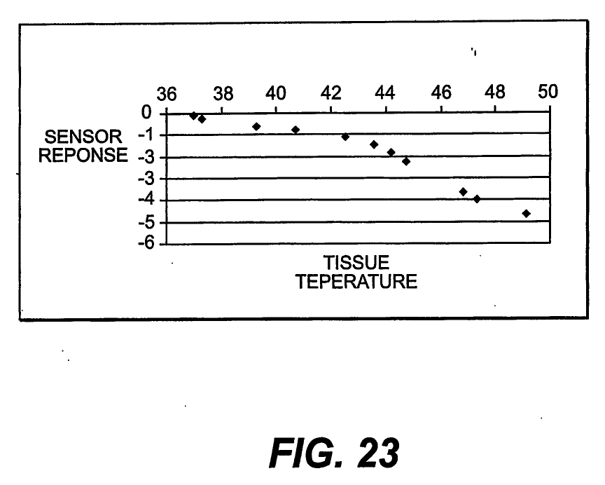

The temperature response of the sensor, operating in CCA mode was determined as a function of the temperature of heated beef tissue in a saline bath containing the RF ablation tip with the embedded sensor. As shown in FIG. 23, that the magnitude response (in Volts) of the over a range of tissue temperatures (degrees Celsius) sensor is approximately linearly proportional to the tissue temperature. The sign of the response is due to the amplification polarity, and is not of physical significance in this case.

While various embodiments of the present invention have been illustrated, those embodiments have been presented by way of examples and are not intended to limit the scope of the present invention. Furthermore, while this present invention is described herein in the context of measuring blood velocity, it will be apparent to those skilled in the art that this invention is also applicable to other types of sensors.

What is claimed is:

1. A device for measuring blood flow in a blood vessel, comprising
a catheter having a curved outer surface; and
at least one conformal blood flow sensor on the curved outer surface, wherein the at least one blood flow sensor is configured to measure the blood flow.

2. A device according to claim 1, wherein
the catheter curved outer surface is a substrate for the formation of the at least one conformal blood flow sensor, and
the at least one conformal blood flow sensor is a microfabricated sensor formed on the substrate, the formation comprising at least one of
using one or more flexible shadow masks to define one or more features on the substrate,
forming one or more conductive traces on the substrate,
forming one or more access openings or pockets on the substrate,
depositing one or more adhesion layers on the substrate,
depositing one or more insulation layers on the substrate,
depositing one or more contact layers on the substrate,
depositing one of more sensor layers on the substrate,
depositing one or more protection layers on the substrate, and
affixing one or more additional substrates to the curved outer surface.

3. A medical or surgical device, comprising:
a portion comprising a curved outer surface configured for at least one medical or surgical application; and
at least one conformal sensor on the curved outer surface, wherein the at least one conformal sensor is configured for measurements in a medical or surgical application, wherein
the curved outer surface is a substrate for the formation of the at least one conformal sensor, and
the at least one conformal sensor is a microfabricated sensor formed on the substrate, the formation comprising at least one of:
(i) using one or more flexible shadow masks to define one or more features on the substrate,
(ii) forming one or more conductive traces on the substrate,
(iii) forming one or more access openings or pockets on the substrate,
(iv) depositing one or more adhesion layers on the substrate,
(v) depositing one or more insulation layers on the substrate,
(vi) depositing one or more contact layers on the substrate,
(vi) depositing one of more sensor layers on the substrate,
(viii) depositing one or more protection layers on the substrate, and
(ix) affixing one or more additional substrates to the curved outer surface.

4. A device according to claim 3, wherein the at least one medical or surgical application is chosen from internal medical and surgical applications.

5. A device according to claim 1, wherein the at least one conformal blood flow sensor comprises at least one thermoresistor element configured to generate heat and optionally at least one thermoresistor element configured to sense temperature,
wherein the at least one conformal blood flow sensor is configured to measure blood flow by generating heat with the at least one heating element and measuring a change in at least one temperature dependent electrical property of at least one of the at least one heating element and the at least one optional temperature sensing element.

6. A device according to claim 5, wherein the at least one thermoresistor element has a thermoresistivity coefficient of greater than 100 ppm per degree Celcius or less than −100 ppm per degree Celcius.

7. A device according to claim 5, further comprising control electronics configured to heat the at least one heating element, measure the change in the at least one temperature dependent electrical property.

8. A device according to claim 5, wherein at least one conformal blood flow sensor is configured to measure blood flow using at least one of constant current anemometry, constant temperature anemometry, and constant voltage anemometry, pulse-width-modulation anemometry, and constant heat flux anemometry.

9. A device according to claim 5, wherein the at least one conformal blood flow sensor is configured to measure a blood flow direction, and wherein the conformal blood flow sensor comprises at least one of
(i) at least a first and a second thermoresistor, wherein the first and second thermoresistor are configured to be alternately used as heating elements and as sensing elements, and
(ii) at least a first, second, and third thermoresistor, wherein the second thermoresistor is configured as a heating element and is positioned between the first and third thermoresistors, which are configured as temperature sensing elements.

10. A device according to claim 1, further comprising at least one ultrasonic sensor configured to measure at least one of density, thickness, and distance.

11. A device according to claim 10, wherein the at least one ultrasonic sensor comprises at least one piezoelectric material chosen from polymeric piezoelectric materials, piezoelectric ceramic materials, and composite piezoelectric material.

12. A device according to claim 1, further comprising at least one strain sensor on the outer curved surface the catheter, and control electronics configured to measure at least one circuit parameter of the strain sensor that is an indicative of local strain in the at least one strain sensor.

13. A device according to claim 1, further comprising at least one temperature sensor.

14. A method for measuring a flow of blood in a blood vessel, comprising:
inserting a device according to claim 5 into the blood vessel;
measure the blood flow by generating heat with the at least one heating element and measuring the change in the temperature dependent electrical property.

15. A device for measuring at least one physical parameter, comprising
a medical or surgical instrument having an outer surface, wherein the instrument is configured for at least one medical or surgical application; and
at least one conformal sensor on the outer surface, wherein the at least one sensor is configured to measure the at least one physical parameter.

16. A device according to claim 15, wherein the instrument is chosen from ablation tips, needles, blades, probes, cannula, forceps, grippers, micro-grippers, endoscopic tools, and end-effector of surgical instruments.

17. A device according to claim 15, wherein the outer surface is chosen from flat surfaces, curved surfaces, or any combination thereof.

18. A device according to claim 15, wherein the at least one physical parameter is chosen from temperature, flow rate, flow direction, density, force, temperature, pH, biochemical composition, location, size, distance, pressure, instrument temperature, instrument location, instrument strain, instrument contact, instrument force, instrument velocity, and instrument acceleration.

19. A device according to claim 15, wherein the at least one conformal sensor was not formed on a semiconductor wafer.

20. A method of forming a conformal sensor on a medical or surgical device having a curved outer surface configured for internal medical or surgical applications, comprising
using the curved outer surface of the device as a substrate for the formation of at least one conformal sensor, and
microfabricating the at least one conformal sensor on the substrate, the microfabricating comprising at least one of:
(i) using at least one flexible shadow mask to define one or more features on the substrate,
(ii) forming at least one conductive traces on the substrate,
(iii) forming at least one access openings or pockets on the substrate,
(iv) depositing at least one adhesion layers on the substrate,
(v) depositing at least one insulation layers on the substrate,
(vi) depositing at least one contact layers on the substrate,
(vii) depositing one of more sensor layers on the substrate,
(viii) depositing at least one protection layers on the substrate, and
(ix) affixing at least one additional substrates to the curved outer surface.

21. A method of forming a conformal blood flow sensor on a catheter, comprising
using a curved outer surface of the catheter curved as a substrate for the formation of at least one conformal blood flow sensor, and
microfabricating the at least one conformal blood flow sensor on the substrate, the microfabricating comprising at least one of:
(i) using at least one flexible shadow mask to define one or more features on the substrate,
(ii) forming at least one conductive traces on the substrate,
(iii) forming at least one access openings or pockets on the substrate,
(iv) depositing at least one adhesion layers on the substrate,
(v) depositing at least one insulation layers on the substrate,
(vi) depositing at least one contact layers on the substrate,
(vii) depositing one of more sensor layers on the substrate,
(viii) depositing at least one protection layers on the substrate, and
(ix) affixing at least one additional substrates to the curved outer surface.

22. A method according to claim 21, wherein the microfabricating comprises the using the at least one flexible shadow mask, positioning the at least one flexible shadow mask to follow a contour of curved substrate surface, and forming one or more features on the substrate though holes in the at least one flexible shadow mask.

23. A method according to claim 22, wherein the at least one flexible shadow mask has a permanent or semi-permanent adhesive layer for attaching the at least one flexible shadow mask to the substrate.

24. A method according to claim 22, wherein the forming the one or more features comprises at least one of a thin film deposition process and a material removal process.

25. A method according to claim 21, wherein the microfabricating comprises forming the at least one adhesion layer by at least one of roughening said substrate and depositing at least one material that strongly adheres to said substrate.

26. A method according to claim 21, wherein the microfabricating comprises forming the at least one contact layer by depositing electrically conductive material.

27. A method according to claim 21, wherein the microfabricating comprises forming the at least one insulation layer by depositing dielectric material sufficient to prevent electrical shorts between electrically conductive layers separated by the at least one insulating layer.

28. A method according to claim 21, wherein the microfabricating comprises forming the at least one protection layer by depositing a material resistant to at least one of moisture and chemicals over at least one other layer.

29. A method according to claim 21, wherein the microfabricating comprises forming the at least one sensor layer by depositing at least one sensor material on the substrate.

30. A method according to claim 21, wherein the microfabricating comprises affixing the at least one additional substrate to the curved outer surface, wherein the at least one additional substrate is a polymer layer sufficiently flexible to conform to the curved outer surface; and
forming at least one sensor on the at least one additional substrate, the sensor forming occurring (i) prior, (ii) subsequent, or (iii) partially prior and partially subsequent to the affixing the at least one additional substrate.
